(19) United States
(12) Patent Application Publication
(10) Pub. No.: US 2018/0104334 A1
(43) Pub. Date: Apr. 19, 2018

(54) STORAGE BUFFER

(71) Applicant: MYCARTIS NV, ZWIJNAARDE (BE)

(72) Inventors: Didier FALCONNET, VUFFLENS-LA-VILLE (CH); Lucienne LAGOPOULOS, BOREX (CH)

(73) Assignee: MYCARTIS NV, ZWIJNAARDE (BE)

(21) Appl. No.: 15/573,188

(22) PCT Filed: May 20, 2016
(86) PCT No.: PCT/EP2016/061400

§ 371 Date: Nov. 10, 2017

(30) Foreign Application Priority Data
May 20, 2015 (EP) 15168524.5

(51) Int. Cl. A61K 39/395

(52) U.S. Cl. CPC A61K 39/39591

(57) ABSTRACT

The present invention relates to a composition for storing at least one biomolecule, the composition comprising: a. between about 0.2% w/w to about 20.0% w/w of D-(+)-Trehalose dehydrate; and b. between about 0.1% w/w to about 10.0% w/w of D-Mannitol; c. between about 0.01% w/w to 0.3% w/w of polyoxyethylenesorbitan monooleate; and d. between about 1.0 nM to about 500 mM of Sodium Citrate at pH between about 4.0 to about 8.0; and e. between strictly more than about 0% w/w to about 0.5% w/w of preservative solution, said preservative solution comprising: i. between about 1.0% w/w and about 5.0% w/w of 5 Chloro-2 methyl-4-isothiazolin-3-one; and ii. between about 0.1% w/w and about 3.0% w/w 2-Methyl-4-isothiazolin-3-one.

The present invention relates to a composition for storing a biomolecule and uses thereof for storing the biomolecule in liquid phase. The invention also relates to a method for storing a biomolecule in solid phase and to a method for storing a biomolecule in liquid phase.

Biomolecules can be in active or inactive state. The active state of a biomolecule is based on covalent and non-covalent interactions between atoms that shape the three dimensional structure of the biomolecule and thereby ensure a functional and operative state of the biomolecule. In contrast, when stored in inappropriate conditions, at least part of non-covalent interactions is altered and the biomolecule turns into inactive state. For instance, enzymes in active state are capable of catalysing biochemical reactions whereas no biochemical reactions can be catalysed when the enzymes are in inactive state.

Performing biological assay commonly requires commercial biomolecules provided by the supplier in an appropriate storage buffer. In this respect, a storage buffer aims at sustaining the biomolecule in an active state. Basically, biomolecules are stored either in solid phase via lyophilisation or in liquid phase. Lyophilisation usually comprises successive freeze drying steps applied to a buffered solution of the active biomolecule. Alternatively, the active biomolecule is diluted in a dedicated storage buffer solution for liquid storage. Generally, solid or liquid phase storage is chosen notably depending on the kind of biomolecule, the stability of the biomolecule and the duration of the storage. Existing storage buffers are generally specifically designed either for liquid phase storage or solid phase storage. To the knowledge of the applicant, there is no polyvalent storage buffer for storing biomolecules in solid and liquid phases.

In the course of optimizing the storage of a product, the applicant developed a unique storage buffer for storing the biomolecule either in solid state or in liquid state.

The present invention fulfills these objectives by providing a composition for storing at least one biomolecule, the composition comprising:
a. between about 0.2% w/w to about 20.0% w/w of D-(+)-Trehalose dehydrate; and
b. between about 0.1% w/w to about 10.0% w/w of D-Mannitol;
c. between about 0.01% w/w to 0.3% w/w of polyoxyethylenesorbitan monooleate; and
d. between about 1.0 nM to about 500 mM of Sodium Citrate at pH between about 4.0 to about 8.0; and
e. between about 0% w/w to about 0.5% w/w of preservative solution, said preservative solution comprising:
   i. between about 1.0% w/w and about 5.0% w/w of 5-Chloro-2 methyl-4-isothiazolin-3-one; and
   ii. between about 0.1% w/w and about 3.0% w/w of 2-Methyl-4-isothiazolin-3-one.

The invention also relates to a method for storing in liquid phase at least one biomolecule, the method comprising the successive steps of:
i. providing a container comprising said biomolecule;
ii. solubilizing said biomolecule with a composition according to the present invention;
iii. filling the container with an inert gas;
iv. sealing the container.

Furthermore, the invention also concerns a method for storing in solid phase at least one biomolecule, the method comprising the successive steps of:
i. providing a container comprising said biomolecule;
ii. solubilizing said biomolecule with a composition according to the present invention;
iii. lyophilizing said biomolecule by applying at least:
   1. a freezing step at a first pressure P1; and
   2. a primary drying step at a second pressure P2, said P2 being lower than said first pressure P1; and
iv. filling the container with an inert gas;
v. sealing the container.

Finally, the invention relates to the use of the composition according to the present invention for storing in liquid phase at least a biomolecule.

Thus, the present invention solves the problem by providing a composition allowing storing at least a biomolecule either in solid or liquid phase. The composition according to the present invention provides flexibility on storage temperature so that the composition can be used for storing biomolecules requiring different storage temperature.

Biomolecules coupled to a solid support may be prone to degradation because of the interaction of the biomolecule with said solid support. However, the composition according to the present invention proves to be efficient to store biomolecules attached to solid support both in liquid state or solid state. Thus the composition proves to be adequate to store proteins coupled to microparticles. When it comes to store biomolecules, the composition according to the present invention outperforms existing storage buffers, either for solid or liquid storage so that proportion of biomolecule in active state remains higher for a longer time in said composition than the proportion of biomolecule active state in existing storage buffers, as illustrated notably in example 3 below in a study over 42 days at −20° C., 4° C., 24° C. and 37° C. Therefore, the composition according to the present invention further allows storing biomolecule, and more particularly capture biomolecules in active state either in liquid state or solid state.

Solid storage, for instance via lyophilisation though freeze drying process typically required several steps at different pressure and temperature. On the contrary, liquid storage process comprises a limited number of steps, in most cases the biomolecule to be stored being simply diluted in the liquid storage composition and the air above the liquid being replaced by an inert gas.

Advantageously, the composition according to the present invention is particularly adapted to store at least a biomolecule in liquid phase. In particular, the composition according to the present invention permits to store biomolecules in active state at room temperature, for instance between 20° C. and 30° C. which is very interesting when it comes to store biomolecules in a laboratory. In this respect, it seems that the presence of saccharide derivatives, for instance the trehalose and the mannitol in the composition according to the present invention, play an important role to preserve the activity of the biomolecule.

According to an embodiment, the composition according to the invention comprises:
a. between about 8.0% w/w to about 10.0% w/w of D-(+)-Trehalose dehydrate; and
b. between about 1.8% w/w to about 2.2% w/w of D-Mannitol; and
c. between about 0.04% w/w to 0.06% w/w of polyoxyethylenesorbitan monooleate; and
d. between about 45 nM to about 55 mM of Sodium Citrate at pH between about 5.5 to about 6.5; and
e. between about 0% w/w to about 0.25% w/w of preservative solution, said preservative solution comprising:
   i. between about 2.1% w/w and about 2.5% w/w of 5-Chloro-2 methyl-4-isothiazolin-3-one; and
   ii. between about 0.6% w/w and about 0.8% w/w 2-Methyl-4-isothiazolin-3-one.

Advantageously, the composition according to this embodiment improves significantly the proportion of biomolecule in active state when compared to existing storage solutions. Moreover, said composition also permits to preserve the biomolecule in active state.

In an embodiment, the composition comprises strictly more than 0% w/w of preservative solution. In other terms, according to said embodiment, the absence of preservative solution is excluded, i.e. the composition of the invention necessarily comprises the said preservative solution.

According to said embodiment, the composition of the invention comprises:
a. between about 0.2% w/w to about 20.0% w/w of D-(+)-Trehalose dehydrate; and
b. between about 0.1% w/w to about 10.0% w/w of D-Mannitol;
c. between about 0.01% w/w to 0.3% w/w of polyoxyethylenesorbitan monooleate; and
d. between about 1.0 nM to about 500 mM of Sodium Citrate at pH between about 4.0 to about 8.0; and
e. between about 0% w/w, with the proviso that this rate is strictly greater than 0, to about 0.5% w/w of a preservative solution, said preservative solution comprising:
   i. between about 1.0% w/w and about 5.0% w/w of 5-Chloro-2 methyl-4-isothiazolin-3-one; and
   ii. between about 0.1% w/w and about 3.0% w/w of 2-Methyl-4-isothiazolin-3-one.

According to said embodiment, the composition of the invention preferably comprises:
a. between about 0.2% w/w to about 20.0% w/w of D-(+)-Trehalose dehydrate; and
b. between about 0.1% w/w to about 10.0% w/w of D-Mannitol;
c. between about 0.01% w/w to 0.3% w/w of polyoxyethylenesorbitan monooleate; and
d. between about 1.0 nM to about 500 mM of Sodium Citrate at pH between about 4.0 to about 8.0; and
e. between strictly more than 0% w/w to about 0.5% w/w of a preservative solution, said preservative solution comprising:
   i. between about 1.0% w/w and about 5.0% w/w of 5-Chloro-2 methyl-4-isothiazolin-3-one; and
   ii. between about 0.1% w/w and about 3.0% w/w of 2-Methyl-4-isothiazolin-3-one.

More preferably, according to this embodiment, the composition of the invention comprises:
a. between about 0.2% w/w to about 20.0% w/w of D-(+)-Trehalose dehydrate; and
b. between about 0.1% w/w to about 10.0% w/w of D-Mannitol;
c. between about 0.01% w/w to 0.3% w/w of polyoxyethylenesorbitan monooleate; and
d. between about 1.0 nM to about 500 mM of Sodium Citrate at pH between about 4.0 to about 8.0; and
e. between about 0.01% w/w to about 0.5% w/w of preservative solution, said preservative solution comprising:
   i. between about 1.0% w/w and about 5.0% w/w of 5-Chloro-2 methyl-4-isothiazolin-3-one; and
   ii. between about 0.1% w/w and about 3.0% w/w of 2-Methyl-4-isothiazolin-3-one.

Yet more preferably, in this embodiment, the preservative solution represents between about 0.05% w/w and about 0.5% w/w of the composition of the invention, more preferably between about 0.05% w/w and about 0.5% w/w, yet more preferably between 0.1% w/w and about 0.5% w/w, said preservative solution comprising:
i. between about 1.0% w/w and about 5.0% w/w of 5-Chloro-2 methyl-4-isothiazolin-3-one; and
ii. between about 0.1% w/w and about 3.0% w/w 2-Methyl-4-isothiazolin-3-one.

Advantageously, according to an embodiment, the composition of the invention comprises:
a. between about 8.0% w/w to about 10.0% w/w of D-(+)-Trehalose dehydrate; and
b. between about 1.8% w/w to about 2.2% w/w of D-Mannitol; and
c. between about 0.04% w/w to 0.06% w/w of polyoxyethylenesorbitan monooleate; and
d. between about 45 nM to about 55 mM of Sodium Citrate at pH between about 5.5 to about 6.5; and
e. between about 0%, with the proviso that this rate is strictly greater than 0, to about 0.25% w/w of preservative solution, said preservative solution comprising:
   i. between about 2.1% w/w and about 2.5% w/w of 5-Chloro-2 methyl-4-isothiazolin-3-one; and
   ii. between about 0.6% w/w and about 0.8% w/w 2-Methyl-4-isothiazolin-3-one.

Advantageously, according to the same embodiment, the composition of the invention preferably comprises:
a. between about 8.0% w/w to about 10.0% w/w of D-(+)-Trehalose dehydrate; and
b. between about 1.8% w/w to about 2.2% w/w of D-Mannitol; and
c. between about 0.04% w/w to 0.06% w/w of polyoxyethylenesorbitan monooleate; and
d. between about 45 nM to about 55 mM of Sodium Citrate at pH between about 5.5 to about 6.5; and
e. between strictly more than 0% to about 0.25% w/w of preservative solution, said preservative solution comprising:
   i. between about 2.1% w/w and about 2.5% w/w of 5-Chloro-2 methyl-4-isothiazolin-3-one; and
   ii. between about 0.6% w/w and about 0.8% w/w 2-Methyl-4-isothiazolin-3-one.

More advantageously, according to the same embodiment, the composition of the invention comprises:
a. between about 8.0% w/w to about 10.0% w/w of D-(+)-Trehalose dehydrate; and
b. between about 1.8% w/w to about 2.2% w/w of D-Mannitol; and
c. between about 0.04% w/w to 0.06% w/w of polyoxyethylenesorbitan monooleate; and
d. between about 45 nM to about 55 mM of Sodium Citrate at pH between about 5.5 to about 6.5; and
e. between about 0.01% w/w to about 0.25% w/w of preservative solution, said preservative solution comprising:
   i. between about 2.1% w/w and about 2.5% w/w of 5-Chloro-2 methyl-4-isothiazolin-3-one; and
   ii. between about 0.6% w/w and about 0.8% w/w 2-Methyl-4-isothiazolin-3-one.

Advantageously, in this embodiment, the preservative solution represents between about 0.05% w/w and about 0.25% w/w of the composition of the invention, more preferably between about 0.05% w/w and about 0.25% w/w, yet more preferably between 0.1% w/w and about 0.25% w/w, said preservative solution comprising:
i. between about 2.1% w/w and about 2.5% w/w of 5-Chloro-2 methyl-4-isothiazolin-3-one; and
ii. between about 0.6% w/w and about 0.8% w/w 2-Methyl-4-isothiazolin-3-one.

In an embodiment, the at least a biomolecule is coupled on a solid support. Advantageously, the composition according to the present invention is suitable for storing biomolecules coupled on at least a solid support. For instance, the present invention is particularly adapted to store antibodies coupled to at least a microparticle.

In one embodiment, a storage buffer for storing at least a biomolecule in liquid and/or solid storage consists of the composition according to the present invention.

In an embodiment, the biomolecule is a protein, preferably an antibody.

In one embodiment, the inert gas is chosen among nitrogen or argon or mixture thereof.

According to the present invention, the terms “storing at least a biomolecule” refers to the storage of a biomolecule after its production in an appropriate media, said media being suitable for limiting the denaturation of the biomolecule.

According to the present invention, the terms “preservative solution” refer to a solution capable of limiting the development of bacteria, and/or fungi and/or yeast in the composition when mixed with said composition.

According to an embodiment, the terms “liquid state” refer to a composition which is essentially in liquid state, preferably in liquid state.

According to the present invention, the terms “storing in liquid phase at least one biomolecule” refers to at least a biomolecule stored on a composition essentially in liquid state, preferably in liquid state.

According to an embodiment, the terms “solid state” refer to a composition which is essentially in solid state, preferably in solid state. The solid state can be obtained by several processes. For instance, said processes are chosen amongst freezing, freeze drying or lyophilisation.

According to the present invention, the terms “storing in solid phase at least one biomolecule” refers to at least a biomolecule stored on a composition essentially in solid state, preferably in solid state.

According to the present invention, the terms “activity of a protein” refers to the relative proportion of active state and inactive state of said protein.

In active state, the protein is functional and operative to perform it function. For instance, if the protein is an enzyme, in active state said enzyme is capable of catalysing biochemical reactions whereas no biochemical reactions can be catalysed when the enzyme are in inactive state.

The present invention is further illustrated by the following detailed description set forth in view of the appended figures, which represent an exemplary and explanatory embodiment of a composition for storing at least one biomolecule and uses thereof. The description comprises the following figures:

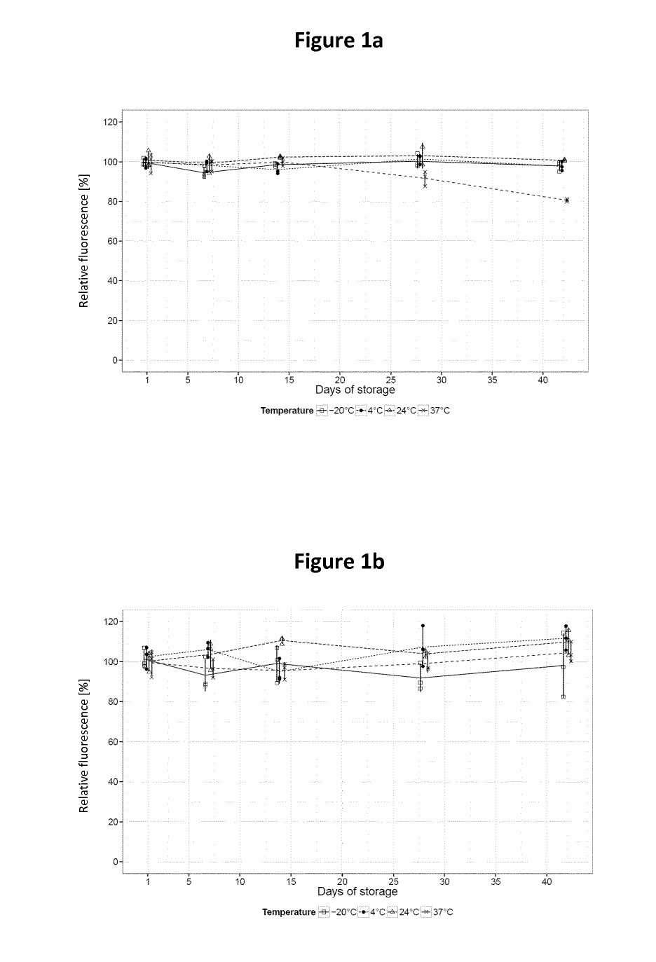

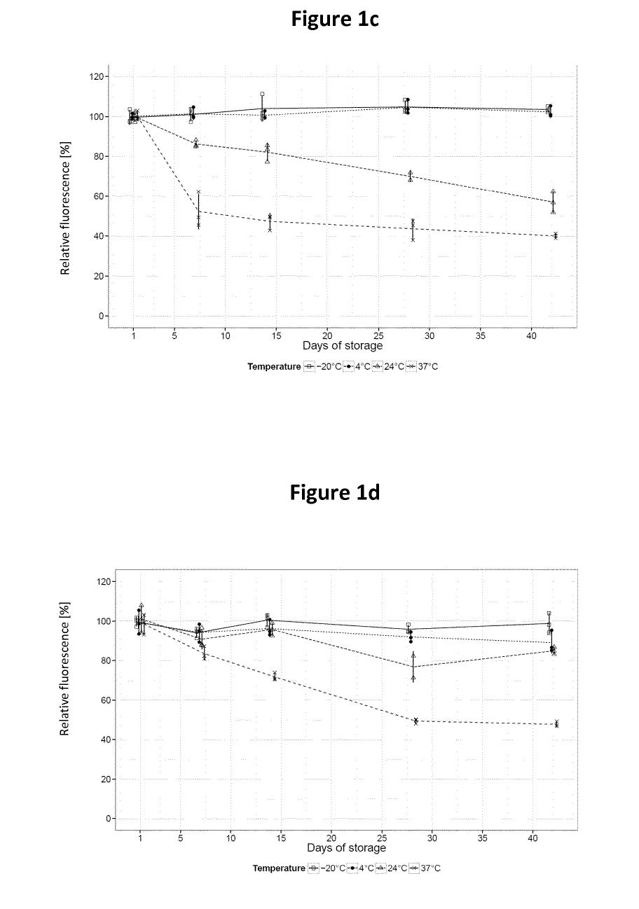

FIG. 1a illustrates the evolution of the fluorescence during immunoassays aiming at detecting capture antibody IL-6 in MCBS buffer over 42 days at −20° C., 4° C., 24° C. and 37° C.;

FIG. 1b illustrates the evolution of the fluorescence during immunoassays aiming at detecting capture antibody TNF-alpha in MCBS buffer over 42 days at −20° C., 4° C., 24° C. and 37° C.;

FIG. 1c illustrates the evolution of the fluorescence during immunoassays aiming at detecting IL-1 beta in MCBS buffer over 42 days at −20° C., 4° C., 24° C. and 37° C.;

FIG. 1d illustrates the evolution of the fluorescence during immunoassays aiming at detecting IL-2 in MCBS buffer over 42 days at −20° C., 4° C., 24° C. and 37° C.;

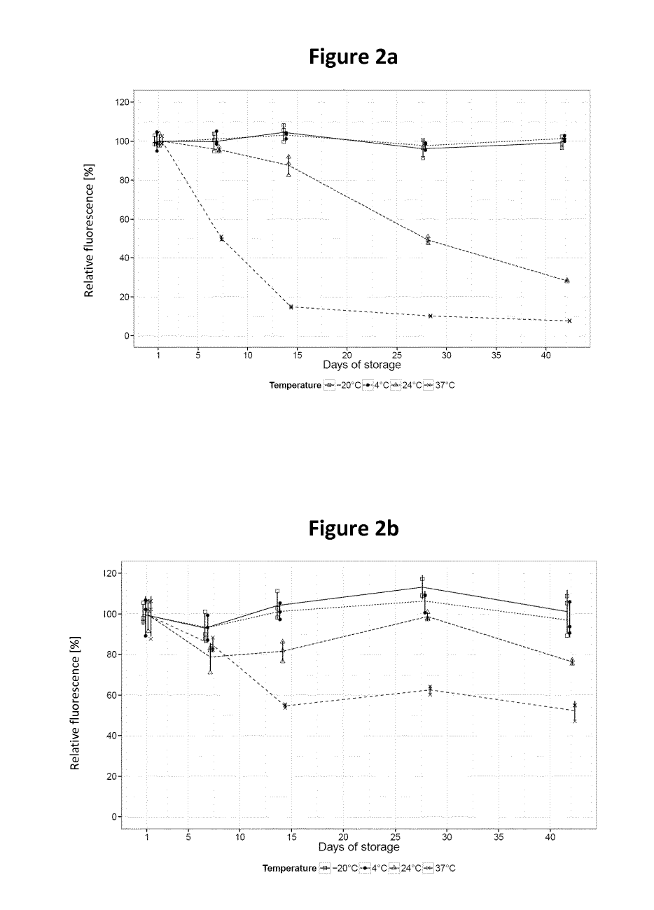

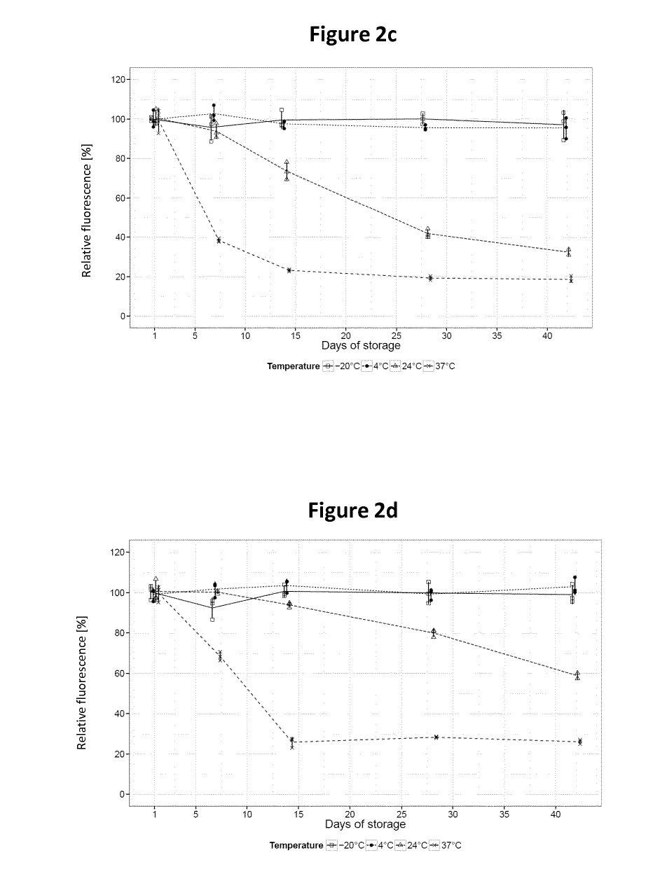

FIG. 2a illustrates the evolution of the fluorescence during immunoassays aiming at detecting capture antibody IL-6 in PBS buffer over 42 days at −20° C., 4° C., 24° C. and 37° C.;

FIG. 2b illustrates the evolution of the fluorescence during immunoassays aiming at detecting capture antibody TNF-alpha in PBS buffer over 42 days at −20° C., 4° C., 24° C. and 37° C.;

FIG. 2c illustrates the evolution of the fluorescence during immunoassays aiming at detecting IL-1 beta in PBS buffer over 42 days at −20° C., 4° C., 24° C. and 37° C.;

FIG. 2d illustrates the evolution of the fluorescence during immunoassays aiming at detecting IL-2 in PBS buffer over 42 days at −20° C., 4° C., 24° C. and 37° C.;

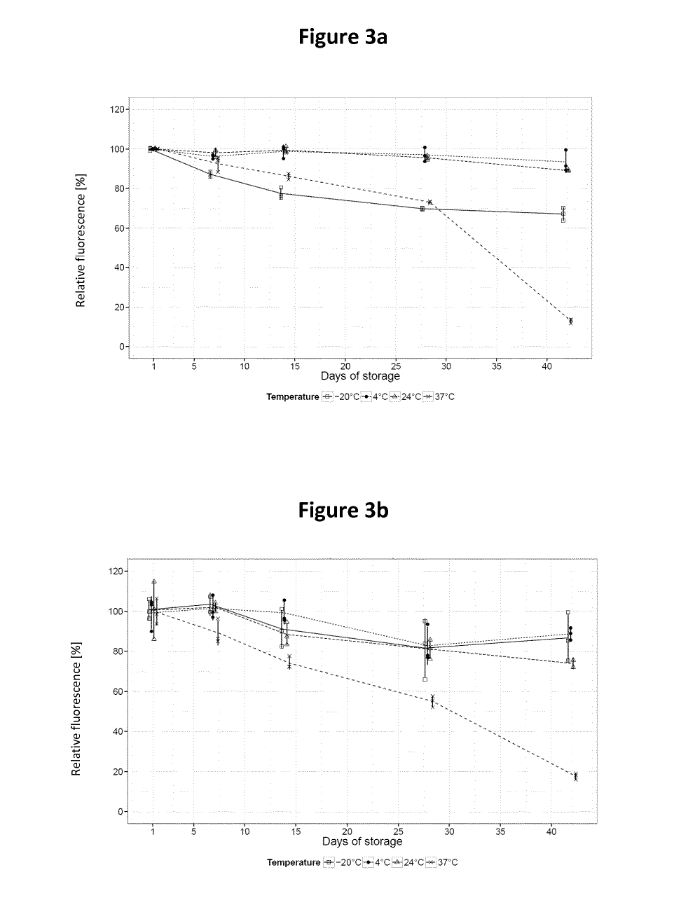

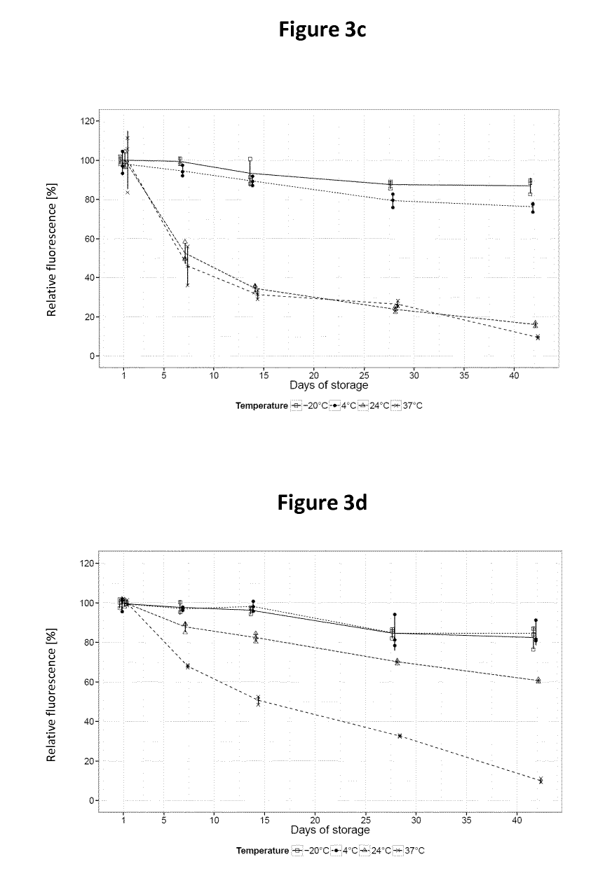

FIG. 3a illustrates the evolution of the fluorescence during immunoassays aiming at detecting capture antibody IL-6 in PBSTNa buffer over 42 days at −20° C., 4° C., 24° C. and 37° C.;

FIG. 3b illustrates the evolution of the fluorescence during immunoassays aiming at detecting capture antibody TNF-alpha in PBSTNa buffer over 42 days at −20° C., 4° C., 24° C. and 37° C.;

FIG. 3c illustrates the evolution of the fluorescence during immunoassays aiming at detecting IL-1 beta in PBSTNa buffer over 42 days at −20° C., 4° C., 24° C. and 37° C.;

FIG. 3d illustrates the evolution of the fluorescence during immunoassays aiming at detecting IL-2 in PBSTNa buffer over 42 days at −20° C., 4° C., 24° C. and 37° C.;

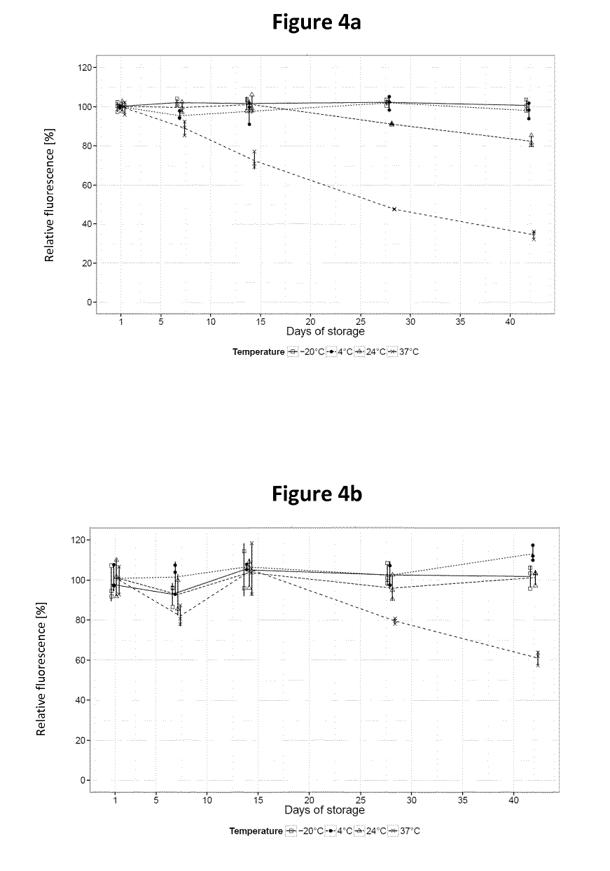

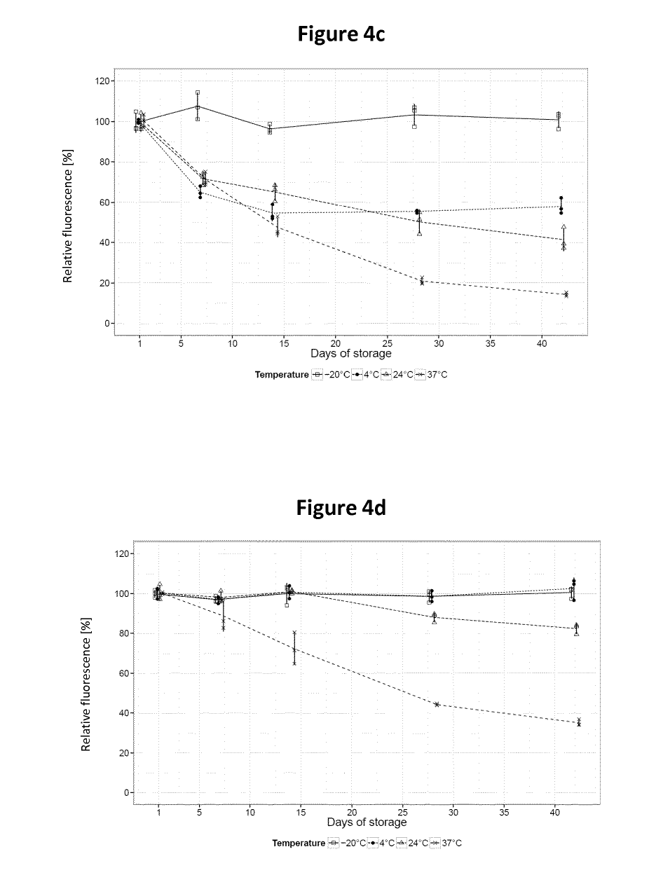

FIG. 4a illustrates the evolution of the fluorescence during immunoassays aiming at detecting capture antibody IL-6 in SG02® (StabilGuard Choice Microarray Stabilizer) buffer over 42 days at −20° C., 4° C., 24° C. and 37° C.;

FIG. 4b illustrates the evolution of the fluorescence during immunoassays aiming at detecting capture antibody TNF-alpha in SG02® (StabilGuard Choice Microarray Stabilizer) buffer over 42 days at −20° C., 4° C., 24° C. and 37° C.;

FIG. 4c illustrates the evolution of the fluorescence during immunoassays aiming at detecting IL-1 beta in SG02® (StabilGuard Choice Microarray Stabilizer) buffer over 42 days at −20° C., 4° C., 24° C. and 37° C.;

FIG. 4d illustrates the evolution of the fluorescence during immunoassays aiming at detecting IL-2 in SG02® (StabilGuard Choice Microarray Stabilizer) buffer over 42 days at −20° C., 4° C., 24° C. and 37° C.

### EXAMPLE 1: PREPARATION OF A STORAGE BUFFER

A storage buffer comprising a composition according to the present invention is prepared by the following steps:

1. Preparing a solution 1 comprising:
i. 5.25 g of citric acid monohydrate (CAS NUMBER 5949-29-1)
ii. 500 ml of water (CAS NUMBER 7732-18-5)

2. Preparing a solution 2 comprising:
i. 29.41 g of sodium citrate dehydrate (CAS NUMBER 6131-04-3)
ii. 2000 ml of water (CAS NUMBER 7732-18-5)

3. Mixing 230 ml of solution 1 and 1770 ml solution 2 to obtain solution 3;

4. Adjusting solution 3 at pH 6 by adding solution 1 and/or solution 2 to solution 3;

5. Preparing solution 4 by mixing solution 3 with:
i. 1.0 g tween 80 (CAS NUMBER 9005-65-6)
ii. 180 g of D-(+)-Trehalose dehydrate (CAS NUMBER 6138-23-4)
iii. 40 g of D-Mannitol (CAS NUMBER 69-65-8)

6. Filtering solution 4 by flowing solution 4 through 0.2 micrometer filter membrane;

7. Preparing solution 5 by mixing solution 4 from step 6 with 2 ml of ProClin® 300 (ProClin® 300 commercialized by Sigma-Aldrich, comprising 5-Chloro-2 methyl-4-isothiazolin-3-one, CAS NUMBER 26172-55-4; and 2-Methyl-4-isothiazolin-3-one, CAS NUMBER 2682-20-4)

The storage buffer prepared herein is suitable for storing at least a biomolecule in liquid and solid phase, preferably in liquid phase.

### EXAMPLE 2: LYOPHILISATION PROCESS FOR LYOPHILIZING BIOMOLECULES COUPLED TO MICROPARTICLES

The Lyophilisation process comprises the following steps:

1. Providing a container comprising the biomolecules or biomolecules grafted on microparticles. For instance, the container can be a test tube or a cartridge. In the present case, the container is a test tube comprising about 20 000 of microparticles;

2. Contacting said microparticles with the appropriate volume of a composition according to the present invention prepared following the example 1 above. In the present example, 3.3 microliter of the composition is dispensed in the test tube, said solution comprising:

3% (w/v) trehalose, 2% (w/v) mannitol, 0.05% Tween 80, 10 mM sodium citrate buffer at pH 6;

3. Lyophilising the microparticles comprised in the composition by applying a freeze drying process comprising:
a freezing step: from room temperature (about 20° C.) to −50° C. over 3 h50 at ambient pressure (about 1 bar); followed by
primary freeze drying step: from −50° C. to 30° C. over 22 at 40 microbar;

4. Contacting the resulting lyophilized microparticles comprised in the container with nitrogen to store them under nitrogen atmosphere;

5. Sealing the container.

### EXAMPLE 3: COMPARISON OF THE ACTIVITY OF FOUR PROTEINS IN FOUR CANDIDATE STORAGE BUFFERS

The present example aims at studying the activity of proteins coupled on microparticles in four candidate storage buffers.

For the present study, the candidate storage buffers are:
MCBS: MyCartis Biomolecule Storage buffer prepared according to example 1 mentioned above;
PBS based buffer (PBS for Phosphate Buffered Saline):
i. The PBS based buffer comprises 10 mM PBS, 0.1% BSA (CAS NUMBER 9048-46-8), 0.02% Tween20 (CAS Number 9005-64-5), 0.05% Sodium azide (CAS Number 26628-22-8);
ii. The pH is adjusted to 7.2-7.4 and said PBS based buffer is filtered on 0.2 micrometer filter membrane;
PBSTNa buffer:
i. The PBSNa buffer comprises 10 mM PBS, 0.3% Tween 20 (CAS Number 9005-64-5), 0.05% Sodium azide (CAS Number 26628-22-8);
ii. The pH is adjusted to 7.2-7.4 and said PBSNa buffer is filtered on 0.2 micrometer filter membrane.
SG02C): StabilGuard Choice Microarray Stabilizer (SG02® commercialized by SurModics, Eden Prairie, Minnesotta USA)

In the present study, two capture antibodies IL-6 (clone MQ2-13A5) and TNF (clone MAb1 commercialized by BD Biosciences®) and two cytokines (IL-1beta and IL-2) were coupled on the microparticles. Prior to the study, the applicant verified that the four proteins can be tested in a multiplex assay format without significant cross-reactivity/non-specific signal.

To determine the proportion of active state of each proteins of interest in each of the candidate storage buffers, the following 7 steps procedure were performed:

1. Coupling one type of protein of interest (chosen amongst capture antibodies IL-6 or TNF and cytokines IL-1 beta and IL-2 in the present example) onto one set of microparticles, each set of microparticles being encoded with a specific code to identify the proteins coupled thereto;

2. Split each set of microparticles in four equal parts and mixing one part in MCBP, one part in PBS based buffer, one part in PBSTNa and one part in SG02®;

6. Determining the proportion of active protein by measuring the fluorescence of each set of the microparticles at each test day in multiplex functional assays; in the present study, the fluorescence measured is emitted by labelled secondary antibodies designed for recognizing the protein of interest coupled to the microparticle.

7. Plot fluorescence of each test day in function of storage time and temperature and normalize the fluorescence to a reference aliquot stored in each candidate storage buffer at −80° C.

In the present example, the two capture antibodies (IL-6 and TNF) and two cytokines (IL-1beta and IL-2) were stored in the candidate buffers at different temperatures and tested in multiplex assays at day 1, 7, 14, 28 and 42 days.

Each storage temperature has a dedicated purpose: samples stored at −80° C. are used as references with the assumption that no degradation occurs and account for the inter-assay variations; results from −80° C. are used as a fluorescence normalization reference at each test day; 20° C. and 4° C. are convenient storage temperatures and are used to assess the proportion of active state in real time; 24° C. and 37° C. serve the purpose of accelerated ageing and allow early evaluation of buffer candidates.

In the present example, the fluorescence was measured at −80° C., −20° C., 4° C., 24° C. and 37° C. During the immunoassays, when the protein coupled to the microparticles is active state, said protein can be detected so that a fluorescent signal is emitted. On the contrary, when said protein is in inactive state, the protein is not detected and no signal is emitted. Therefore, the measured fluorescence allows estimating the relative proportions of biomolecule in active state and in inactive state.

When capture antibodies IL-6 and TNF-alpha are stored in extreme conditions, i.e. 37° C. for 42 days, losses of 40 to 90% of active state are observed, except when stored in MCPS buffer where the loss in active state is limited to −20%. Therefore, MCPS does not interfere with the activity of capture antibodies IL-6 and THF alpha. MCPS fully preserves the active state of the two antibodies tested contrary to the other candidate buffers. When stored at 24° C. for 42 days, the loss of active state of both capture antibodies IL-6 and TNF alpha is very limited in MCBS whereas it is not the case for the other candidate storage buffers. Moreover, data at 4° C. or −20° C. did not show any significant loss of active state after 42 days in MCBP for the capture antibodies.

Either at −20° C., 4° C. or 24° C., no loss of active state is observed for the capture antibodies in MCBP which is not the case in the others candidate storage buffers. Therefore, MCBP allow storing capture antibodies in active state either in liquid state or solid state contrary to the other candidate storage buffers. These findings are demonstrated in the present study for storage of capture antibodies in liquid phase at 4° C., 24° C. and 37° C. and for storage in solid state at −80° C. and −20° C.

Regarding cytokines, the loss of active state for IL-2 is limited to 50% in MCBP which is not the case in the others storage candidate buffers. The data also indicate that at 37° C. and 24° C., the proportion of IL1-beta in active state is more important in MCBS compared to the proportion of IL1-beta in active state in the other candidate storage buffers. When the coupled cytokines are stored at 4° C. or below in MCPS buffer no significant loss of active state is observed after 42 days for both cytokines contrary to the other candidate buffers.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. Composition for storing at least one biomolecule, the composition comprising:
a. between about 0.2% w/w to about 20.0% w/w of D-(+)-Trehalose dehydrate;
b. between about 0.1% w/w to about 10.0% w/w of D-Mannitol;
c. between about 0.01% w/w to 0.3% w/w of polyoxyethylenesorbitan monooleate;
d. between about 1.0 nM to about 500 mM of Sodium Citrate at pH between about 4.0 to about 8.0; and
e. between strictly more than 0% w/w to about 0.5% w/w of preservative solution, said preservative solution comprising:
i. between about 1.0% w/w and about 5.0% w/w of 5 Chloro-2 methyl-4-isothiazolin-3-one; and
ii. between about 0.1% w/w and about 3.0% w/w 2-Methyl-4-isothiazolin-3-one.

2. Composition according to claim 1, comprising:
a. between about 8.0% w/w to about 10.0% w/w of D-(+)-Trehalose dehydrate;
b. between about 1.8% w/w to about 2.2% w/w of D-Mannitol;
c. between about 0.04% w/w to 0.06% w/w of polyoxyethylenesorbitan monooleate;
d. between about 45 nM to about 55 mM of Sodium Citrate at pH between about 5.5 to about 6.5; and
e. between strictly more than 0% w/w to about 0.25% w/w of preservative solution, said preservative solution comprising:
i. between about 2.1% w/w and about 2.5% w/w of 5-Chloro-2 methyl-4 isothiazolin-3-one; and
ii. between about 0.6% w/w and about 0.8% w/w 2-Methyl-4-isothiazolin-3-one.

3. Composition according to claim 1, comprising:
a. between about 0.2% w/w to about 20.0% w/w of D-(+)-Trehalose dehydrate;
b. between about 0.1% w/w to about 10.0% w/w of D-Mannitol;
c. between about 0.01% w/w to 0.3% w/w of polyoxyethylenesorbitan monooleate;
d. between about 1.0 nM to about 500 mM of Sodium Citrate at pH between about 4.0 to about 8.0; and
e. between about 0.01% w/w to about 0.5% w/w of preservative solution, said preservative solution comprising:
i. between about 1.0% w/w and about 5.0% w/w of 5-Chloro-2 methyl-4-isothiazolin-3-one; and
ii. between about 0.1% w/w and about 3.0% w/w of 2-Methyl-4-isothiazolin-3-one.

4. Composition according to claim 2, comprising:
a. between about 8.0% w/w to about 10.0% w/w of D-(+)-Trehalose dehydrate;
b. between about 1.8% w/w to about 2.2% w/w of D-Mannitol;
c. between about 0.04% w/w to 0.06% w/w of polyoxyethylenesorbitan monooleate;
d. between about 45 nM to about 55 mM of Sodium Citrate at pH between about 5.5 to about 6.5; and
e. between about 0.01% w/w to about 0.25% w/w of preservative solution, said preservative solution comprising:
i. between about 2.1% w/w and about 2.5% w/w of 5-Chloro-2 methyl-4-isothiazolin-3-one; and
ii. between about 0.6% w/w and about 0.8% w/w 2-Methyl-4-isothiazolin-3-one.

5. Method for storing in liquid phase at least one biomolecule, the method comprising the successive steps of:
i. providing a container comprising said biomolecule;
ii. solubilizing said biomolecule with a composition for storing at least one biomolecule, the composition comprising:
a. between about 0.2% w/w to about 20.0% w/w of D-(+)-Trehalose dehydrate;
b. between about 0.1% w/w to about 10.0% w/w of D-Mannitol;
c. between about 0.01% w/w to 0.3% w/w of polyoxyethylenesorbitan monooleate;
d. between about 1.0 nM to about 500 mM of Sodium Citrate at pH between about 4.0 to about 8.0; and
e. between strictly more than 0% w/w to about 0.5% w/w of preservative solution, said preservative solution comprising:
1. between about 1.0% w/w and about 5.0% w/w of 5 Chloro-2 methyl-4-isothiazolin-3-one; and
2. between about 0.1% w/w and about 3.0% w/w 2-Methyl-4-isothiazolin-3-one;
iii. filling the container with an inert gas; and
iv. sealing the container.

6. Method for storing in solid phase at least one biomolecule, the method comprising the successive steps of:
i. providing a container comprising said biomolecule;
ii. solubilizing said biomolecule with a composition for storing at least one biomolecule, the composition comprising:
a. between about 0.2% w/w to about 20.0% w/w of D-(+)-Trehalose dehydrate;
b. between about 0.1% w/w to about 10.0% w/w of D-Mannitol;
c. between about 0.01% w/w to 0.3% w/w of polyoxyethylenesorbitan monooleate;
d. between about 1.0 nM to about 500 mM of Sodium Citrate at pH between about 4.0 to about 8.0; and
e. between strictly more than 0% w/w to about 0.5 w/w of preservative solution, said preservative solution comprising:
1. between about 1.0% w/w and about 5.0% w/w of 5 Chloro-2 methyl-4-isothiazolin-3-one; and
2. between about 0.1% w/w and about 3.0% w/w 2-Methyl-4-isothiazolin-3-one;
iii. lyophilizing said biomolecule by applying at least:
1. a freezing step at a first pressure P1; and
2. a primary drying step at a second pressure P2, said P2 being lower than said first pressure P1;
iv. filling the container with an inert gas; and
v. sealing the container.

7. Use of the composition according to claim 1 for storing in liquid phase at least a biomolecule.
